(19) United States
(12) Patent Application Publication
(10) Pub. No.: US 2024/0161289 A1
(43) Pub. Date: May 16, 2024

(54) DEFORMABLE IMAGE REGISTRATION USING MACHINE LEARNING AND MATHEMATICAL METHODS

(71) Applicants: GEORGIA TECH RESEARCH CORPORATION, Atlanta, GA (US); Emory University, Atlanta, GA (US)

(72) Inventors: Mohammad Khan, Atlanta, GA (US); Anees Dhabaan, Atlanta, GA (US); Shaheen Dewiji, Atlanta, GA (US); David Gonzalez, Atlanta, GA (US); Ignacio Bartol, Atlanta, GA (US); Samuel Taylor, Atlanta, GA (US)

(21) Appl. No.: 18/503,617

(22) Filed: Nov. 7, 2023

Related U.S. Application Data
(60) Provisional application No. 63/423,113, filed on Nov. 7, 2022.

(51) Int. Cl. G06T 7/00; G16H 20/00; G16H 30/40

(52) U.S. Cl. CPC G06T 7/0012; G16H 20/00; G16H 30/40; G06T 2207/20081; G06T 2207/20116; G06T 2207/30096

(57) ABSTRACT

Disclosed herein is a system and method for deformable image registration of medical image scans. The system and method described herein relate to improving treatment plans for tumors in locations that are susceptible to natural body movements, in particular, to designing radiation treatment as it relates to the respiratory cycle. The exemplary method and system disclosed herein provides a solution to this problem, which reduces the risk of treatment-related side effects and provides a better framework for more aggressive treatment methods where greater accuracy in distribution is required.

### CROSS-REFERENCE TO RELATED APPLICATIONS

This application claims the benefit of priority to U.S. Provisional Application No. 63/423,113, filed Nov. 7, 2022, which is incorporated herein by reference in its entirety.

### TECHNICAL FIELD

This disclosure generally relates to deformable image registration of medical scan images.

### BACKGROUND

The application of Artificial Intelligence (AI) subsets such as Machine Learning (ML) accelerate patient-specific radiation oncology planning and treatment [5] [6] [7]. One of the primary limitations in the radiotherapy workflow was that a single-patient treatment plan is time-consuming to do and may not fully encompass the specific needs of the patient nor leverage all the potential that some radiation therapy methods, such as Adaptive Radiation Therapy (ART) [8] and SBRT [9], can offer. In several areas, AI has been implemented and is a powerful tool helping medical physicists and physicians automate portions of the workflow [10]. One such portion is the automation of the delineation of tumors and surrounding organs. The precise delineation of tumor volumes in an efficient and generalized manner is still a challenge in SBRT planning [11], especially for institutions that cannot perform DIR.

A significant challenge in the reliable delineation of tumor volumes is the movement-induced geometric uncertainties from patient-specific respiratory patterns [12]. Such uncertainties can result in the GTV, the clinical target volume (CTV), the planning target volume (PTV), and OARs changing in volumetric shape and centroidal location throughout the respiratory cycle. 4D-CT scans have aided in characterizing such uncertainties. 4D-CT has been widely used in radiation therapy to track the motion of the GTV and OARs in time [13] [14]. The information offered by such an image modality includes the motion boundaries and trajectories of the tumor, healthy tissue, and other organs, which can improve treatment planning and accuracy of tumor delineation along the different respiratory phases.

Patients who suffer from NSCLC face significant challenges in effective irradiation that adequately covers the target region while sparing the neighboring organs due to the motion of abdominal tissues caused by respiration [15] [16]. To minimize incidental dose to OARs, it is crucial to use DIR methods that are both fast and accurate and that leverage augmented information from 4D-CT scans [17]. However, traditional DIR techniques, such as Horn-Schunk optical flow [1], DEMONS [2], and B-spline-based algorithms [3], are not the most efficient, mainly when working with the typically large 4D-CT datasets [4]. Despite this challenge, researchers continue to develop and improve DIR methods to address the unique challenges of NSCLC treatment planning [18] [19] [20]. The goal is to reconcile the time-varying anatomical landscape with clinically acceptable dose delivery, which includes target definition, GTV tracking, OAR sparing, and respiratory gating. Previous work by Peroni M. et al. [14] proposed an intensity-based B-Spline deformable registration of CT volumes that minimized differences in intensity between images and mapped anatomic contours across images. A 3D B-Spline deformation was employed to map anatomic structures between the reference and target images; in a two-part study evaluating automatic segmentation methods, the NCAT Phantom study revealed limitations due to partial volume interpolation and homogeneous grey levels, impacting deformable registration accuracy. Variations in lesion diameter and Dice Similarity Coefficients (DSC) coefficient values were moderate, ranging from 3.4-5.1% and 0.92-0.93%, respectively. In their patient retrospective study, automatic methods were within 2% of inter-rater variability in DSC, suggesting their clinical applicability.

Moreover, the study by Zheng C. et al. improved a conventional DIR method to account for local rigidity preservation that disallows excessive deformation of the tumor, as opposed to the results that current DIR methods sometimes provide, aimed at preserving tumor-specific features while ensuring accurate global alignment of temporal or multimodal biomedical images. This excessive deformation may mislead the radiation therapy planning, resulting in inaccurate dose values delivered to the tumor and healthy tissue. The study employed twelve clinical datasets with fifteen tumors and considered Positron Emission Tomography (PET) and CT scans for evaluation. The model effectively mitigates excessive tumor deformation, a factor that could compromise treatment accuracy. Quantitative measurements confirmed the model's ability to maintain the integrity of tumor size and shape, particularly in PET-PET and CT-CT registration scenarios.

Lastly, Guy C. L. et al. [20] developed a DIR algorithm called Consistent Anatomy in Lung Parametric imagE Registration (CALIPER), specifically designed to manage substantial geometric changes in the thorax, such as those encountered in cases of atelectasis resolution during ART for NSCLC. Using a hybrid intensity-based and feature-based similarity cost function, the algorithm demonstrated high registration accuracy across varying degrees of atelectasis. Notably, landmark-based target registration error and DSC for atelectatic lobes indicated that CALIPER achieved accuracy levels comparable to state-of-the-art algorithms.

Stereotactic body radiotherapy (SBRT) delivers a highly conformal and hypo fractionated radiation dose to a small target with minimal radiation applied to the surrounding areas. Intensity modulation techniques (IMRT) allows for the radiation dose to conform more precisely to the shape of the tumor by modulating the intensity of the radiation beam and allows higher radiation doses to be focused to regions within the tumor, sparing the surrounding normal regions. Essential for SBRT is precise targeting of the radiation and patient positioning verification. A non-optimized method to achieve this targeting and positioning is not reproducible, is inconsistent, and relies on physician's subjective judgement.

4D computed tomography represents the next step in imaging. This advanced imaging method makes CT more accurate than before by associating a time dependency to the spatial distribution of the area of interest for treatment. It uses a new technology to capture the location and movement of the tumor and surrounding organs over time. This is important for accurately treating tumors located on or near organs that move, such as the lungs during the respiratory (breathing) cycle. With traditional imaging, the oncologist only knows the position of a tumor at one point in the respiratory cycle. This method aims ionizing radiation at a location using only the information obtained during 1 CT scan; however, the position of a tumor may change due to involuntary movements of the patient, such as in the torso during the respiratory cycle. The beam of ionizing radiation used to treat the patient may collimate to an area that, on average, represents healthy tissue within the patient, thus causing unnecessary dose.

### SUMMARY

The system and methods described herein relate to improving treatment plans for tumors in locations that are susceptible to natural body movements, in particular, to designing radiation treatment as it relates to the respiratory cycle. The exemplary method and system disclosed herein provides a solution to this problem, which reduces the risk of treatment-related side effects and provides a better framework for more aggressive treatment methods where greater accuracy in distribution is required.

Rather than the mostly manual and slow/iterative process, the disclosed system and method create more accurate delineations through machine learning models, decreasing time spent per patient plan, and apply a more mathematically rigorous and localized manner of selecting the optimal gating window for treatment.

In some aspects, the techniques described herein relate to a method for deformable image registration, the method including: receiving at least one set of medical images for a patient, wherein at least one medical image includes pre-drawn contours; delineating (e.g., clustering) one or more regions of interest in the at least one set of medical images using a trained clustering machine learning algorithm; determining deformed contours for the one or more regions of interest (e.g., tumor) in the at least one set of medical images using a finite element analysis algorithm over one or more of a plurality of phases of a physiological cycle (e.g., phases of breath cycle); and outputting the deformed contours relative to the one or more regions of interest.

In some aspects, the techniques described herein relate to a method, wherein delineating (e.g. clustering) one or more regions of interest in the at least one set of medical images using a trained clustering machine learning algorithm includes: initializing cluster parameters; training a cluster machine learning algorithm using the at least one medical image including pre-drawn contours; delineating a plurality of contours of the one or more regions of interest (e.g., clustering); and clustering voxels of a medical scan image of the second set within the one or more regions of interest.

In some aspects, the techniques described herein relate to a method, wherein the trained clustering machine learning algorithm includes an unsupervised or supervised machine learning algorithm.

In some aspects, the techniques described herein relate to a method, wherein the trained clustering machine learning algorithm includes a Gaussian mixture model.

In some aspects, the techniques described herein relate to a method, wherein determining deformed contours for the at least one set of medical images includes: deriving a deformation field using the finite element analysis algorithm, wherein the deformation field is dependent on an adjustable parameter (i.e., k); and applying the deformation field over the delineated one or more regions of interest over one or more of a plurality of phases of the physiological cycle, thereby forming deformed contours.

In some aspects, the techniques described herein relate to a method, the method further including receiving an adjusted value of the adjustable parameter from a user.

In some aspects, the techniques described herein relate to a method, wherein in response to the user adjusting the adjustable parameter, the deformed contours are re-evaluated with the adjusted parameter (e.g. k′) and the re-evaluated deformed contours are output.

In some aspects, the techniques described herein relate to a method, wherein the finite element analysis algorithm includes a modified Poisson's equation, Navier-Stokes equation tailored for fluid-like tissue behavior, a Lagrangian mechanics-based algorithm to account for energy conservation, an elastodynamic equation for capturing time-dependent deformations.

In some aspects, the techniques described herein relate to a method, further including determining a dose distribution for one or more of the plurality of phases, wherein determining the dose distribution includes: determining a dose distribution for fewer than the plurality of phases; deriving dose parameters from the fewer than the plurality of phases dose distribution; and applying the dose parameters to deformed distribution for the plurality of phases.

In some aspects, the techniques described herein relate to a method, further including determining a dose distribution for one or more of a plurality of phases includes.

In some aspects, the techniques described herein relate to a method, wherein medical images include 4D CT, 4D PET CT, MRI, ultrasound, radionuclide imaging, optical imaging.

In some aspects, the techniques described herein relate to a method, wherein the deformation contours are subsequently used for diagnostic and/or treatment planning.

In some aspects, the techniques described herein relate to a system for deformable image registration, wherein the system includes: a user device including a means for input and output; and one or more computing systems including one or more processors and one or more storage devices, wherein the one or more storage devices have instructions stored thereon, that when executed by the one or more processors, causes the one or more processors to perform a method, the method including: receiving at least one set of medical images for a patient, wherein at least one medical image includes pre-drawn contours; delineating (e.g., clustering) one or more regions of interest in the at least one set of medical images using a trained clustering machine learning algorithm; determining deformed contours for the one or more regions of interest (e.g., tumor) in the at least one set of medical images using a finite element analysis algorithm over one or more of a plurality of phases of a physiological cycle (e.g. phases of breath cycle); and outputting the deformed contours relative to the one or more regions of interest;

In some aspects, the techniques described herein relate to a system, wherein medical images include 4D CT, 4D PET CT, MRI, ultrasound, radionuclide imaging, optical imaging.

In some aspects, the techniques described herein relate to a system, wherein delineating (e.g. clustering) one or more regions of interest in the at least one set of medical images using a trained clustering machine learning algorithm includes: initializing cluster parameters; training a cluster machine learning algorithm using the at least one medical image including pre-drawn contours; delineating a plurality of contours of one or more regions of interest (e.g., clustering); and clustering voxels of a medical image of the at least one set of medical images within the one or more regions of interest.

In some aspects, the techniques described herein relate to a system, wherein the trained clustering machine learning algorithm is a Gaussian mixture model.

In some aspects, the techniques described herein relate to a system, wherein determining deformed contours for the at least one set of medical images includes: deriving a deformation field using the finite element analysis algorithm, wherein the deformation field is dependent on an adjustable parameter (i.e., k); and applying the deformation field over the delineated one or more regions of interest over one or more of a plurality of phases of the physiological cycle thereby forming deformed contours.

In some aspects, the techniques described herein relate to a system, the method further including receiving an adjusted value of the adjustable parameter from a user.

In some aspects, the techniques described herein relate to a system, wherein in response to the user adjusting the adjustable parameter, the deformed contours are re-evaluated with the adjusted parameter (e.g. k′) and the re-evaluated deformed contours are output.

In some aspects, the techniques described herein relate to a system, wherein the finite element analysis algorithm includes a modified Poisson's equation .

### BRIEF DESCRIPTION OF THE DRAWINGS

FIG. 1 shows a schematic of a workflow of an exemplary method.

FIG. 2 shows output from the exemplary method.

FIGS. 3A and 3B show schematics of an exemplary generalized algorithm.

FIG. 4 shows an illustrative embodiment of an exemplary system.

FIG. 5 shows a schematic of a workflow of an exemplary method.

FIG. 6 shows training iterations implemented on a single averaged slice for Case 1 with pseudo-randomly generated initial clusters (top Gaussian distributions) and the results of training (bottom Gaussian distributions) with the accompanying GMM segmentation of the averaged slice.

FIG. 7A illustrates, on the left, both the original deformation field and the contour targeted for deformation are displayed, and the right side illustrates the issue when applying the transformation solely to the contour points, as multiple points belonging to the contour converge to the same final point after the transformation, resulting in a non-closed contour.

FIG. 7B illustrates the contour along with its interior points, such that, when the transformation is applied, the entire solid mass undergoes deformation, yielding a more precise representation of how the contour should be propagated under the given vector field.

FIG. 8 shows visual output throughout the algorithm workflow in an actual patient and implementations. The process begins with a 4D-CT scan of a patient and the corresponding contours (GTV, ITV, etc.). In step A, the body is isolated from the rest of the image, followed by applying the GMM algorithm. Step B involves delineating the ROI encompassing the GTV, with subsequent steps applied to this region. Step C consists of obtaining the GTV's interior points from the manually drawn contours. At the same time, Step D identifies the desired phase to which the contours are to be propagated. Step E illustrates the difference between the fixed (MaxIP phase) and moving (n-th phase) CT scans. Subsequently, in step F, the diffusion coefficient field is acquired using 8. Step G involves solving Poisson's equation to obtain the deformation field multiplied by a proportionality constant k. Finally, Step H applies the deformation field to the set of points within the GTV to achieve the contour propagated to the desired respiratory phase.

FIG. 9 shows the evolution of the DSC while changing the proportionality constant k of the deformation field.

FIG. 10 shows a comparison of a manually drawn contour on the MaxIP, deformed to Phase 6 of the respiratory phase in a single slice. A comparison between the DEMONS algorithms and the in-house developed Poisson+GMM algorithm is presented on the right. The bottom left detail highlights the tumor on both the MaxIP and Phase 6.

FIG. 11 shows Case 3 which has a substantial portion of the GTV embedded in the Upper Chest Wall.

FIG. 12 shows a comparison of performance between DEMONS DIR and the GMM+Poisson's equation DIR. Both algorithms were evaluated on five cases. Four out of the five cases are depicted here for visualization purposes. The contour, manually delineated in the MaxIP, was propagated to the phase exhibiting the greatest difference from the MaxIP projection. The selection criteria for displaying the slices encompassed the tumor's upper third, middle, and lower third, with the upper part being closer to the head.

FIG. 13 shows a plot of the DSC between the DEMONS DIR propagated gross tumor volumes and the GMM+Poisson propagated gross tumor volumes along with the volume for each method plotted individually for Case 1.

FIG. 14 shows three-dimensional renderings of the voxels encompassed by each the DEMONS DIR and GMM+Poisson DIR propagated GTVs for Case 1.

### DETAILED SPECIFICATION

To facilitate an understanding of the principles and features of various embodiments of the present invention, they are explained hereinafter with reference to their implementation in illustrative embodiments.

The system and methods described herein relate to improving treatment plans for tumors in locations that are susceptible to natural body movements, in particular, to designing radiation treatment as it relates to the respiratory cycle. The exemplary method and system disclosed herein provides a solution to this problem, which reduces the risk of treatment-related side effects and provides a better framework for more aggressive treatment methods where greater accuracy in distribution is required.

Rather than the mostly manual and slow/iterative process, the disclosed system and method create more accurate delineations through machine learning models, decreasing time spent per patient plan, and apply a more mathematically rigorous and localized manner of selecting the optimal gating window for treatment.

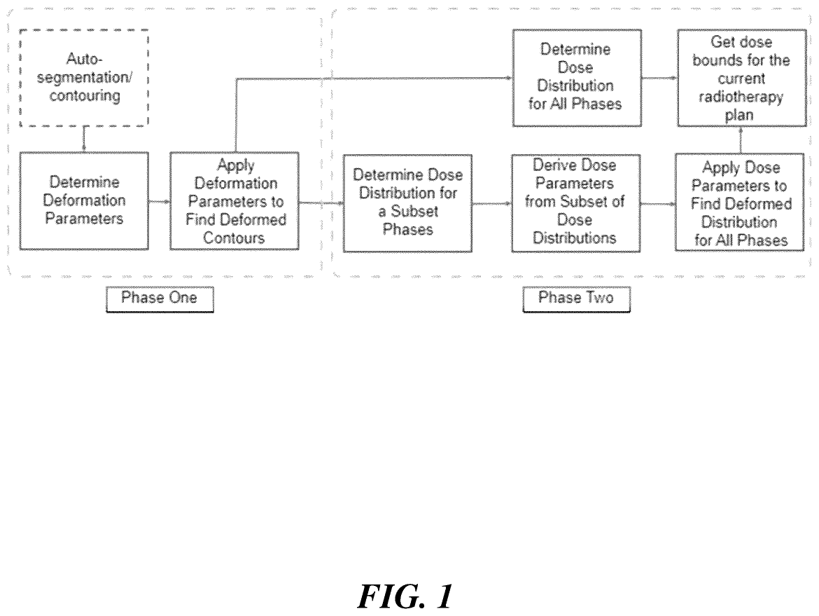

The disclosed method can be understood as having two phases, as shown in FIG. 1. Phase One includes deriving the deformation parameters to describe the three-dimensional movement of a patient's target treatment region through deformation propagation. Phase Two involves a surrogate model for fast reconstruction of the dose distribution in the gross tumor volume (GTV), and organs at risk (OAR) across all respiratory phases, accounting for the deformed target region over time. With these dose profiles, the physicians has the bounds (within a confidence interval) for the absorbed dose by different organs and tumors across all the respiratory cycle phases and will be able to determine if the treatment plan for that patient is accurate and appropriate or if it needs to be replanned. In Phase Two, in addition to higher fidelity three-dimensional time-dependent consideration of GTV and OAR, the algorithm will be translated for clinical use through the development of live, in situ, adaptive treatment during the actual administration of therapy. Dose estimation has traditionally been employed using point kernel or Monte Carlo radiation transport methods. Phase Two further leverages a machine-learning based approach for absorbed dose estimation for the deformable GTV and OAR, thereby developing a fully in situ adaptive treatment planning and administration modality.

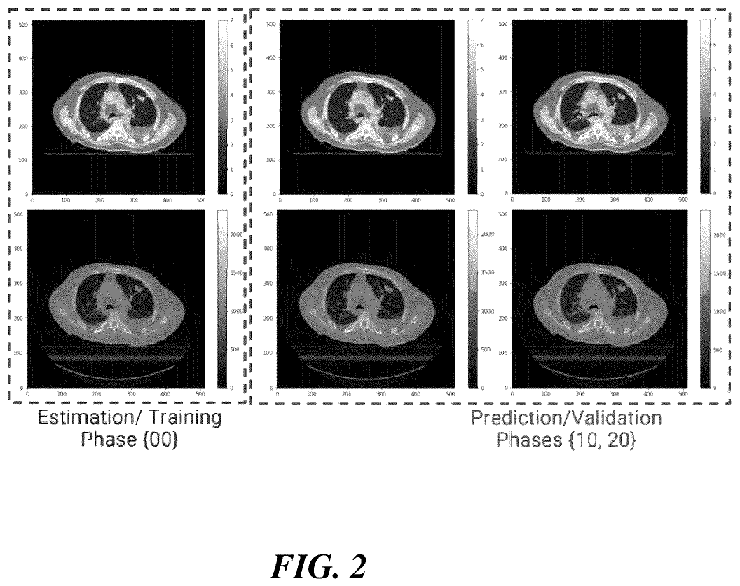

FIG. 2 shows an example output at each phase of the exemplary method.

An exemplary method relates to deformable image registration, the method including: receiving at least one set of medical images for a patient, wherein at least one medical image comprises pre-drawn contours; delineating (e.g., clustering) one or more regions of interest (ROI) in the at least one set medical images using a trained clustering machine learning algorithm; determining deformed contours for the one or more regions of interest (e.g. tumor) in the at least one set of medical images using a finite element analysis algorithm over one or more of a plurality of phases of a physiological cycle (e.g. phases of breath cycle); and outputting the deformed contours relative to the one or more regions of interest. In some embodiment, the deformed contours are subsequently used for diagnostic and/or treatment planning.

In some aspects, the medical images include 4D CT, 4D PET CT, MRI, ultrasound, radionuclide imaging, or optical imaging. In some aspects, treatment planning includes a radiology treatment plan.

Wherein, in some aspects of the exemplary method, delineating (e.g., clustering) one or more ROI in at least one set of medical images using a trained clustering machine learning algorithm includes: initializing cluster parameters; training a cluster machine learning algorithm using the at least one medical image comprising pre-drawn contours; and delineating a plurality of contours of the one or more regions of interest; and clustering voxels of a medical scan image of the second set within the one or more regions of interest. The trained clustering machine learning algorithm includes an unsupervised or supervised machine learning algorithm. In some examples, the trained clustering machine learning algorithm includes a Gaussian mixture model (GMM).

Wherein, in some aspects, determining deformed contours for the at least one set of medical images includes: deriving a deformation field using the finite element analysis algorithm, wherein the deformation field is dependent on an adjustable parameter (i.e., k); applying the deformation field over the delineated one or more ROI over one or more of a plurality of phases of the physiological cycle, thereby forming deformed contours.

In some aspects, the finite element analysis algorithm includes a modified Poisson's equation, a modified Navier-Stokes equation, a modified Lagrangian mechanics-based algorithm, or an elastodynamic equation. The finite element analysis algorithm can evaluate linear and non-linear systems.

In some aspects, the method further includes receiving an adjusted value of the adjustable parameter from a user, wherein in response to the user adjusting the adjustable parameter, the deformed contours are re-evaluated with the adjusted parameter (e.g. k′) and the re-evaluated deformed contours are output.

Wherein, in some aspects, determining the dose distribution for one or more of the plurality of phases includes: determining a dose distribution for fewer than the plurality of phases; deriving dose parameters from the fewer than the plurality of phases dose distribution; and applying the dose parameters to deformed distribution for the plurality of phases.

Whereas, in other aspects, determining the dose distribution for one or more of a plurality of phases includes: determining the dose distribution for the plurality of phases.

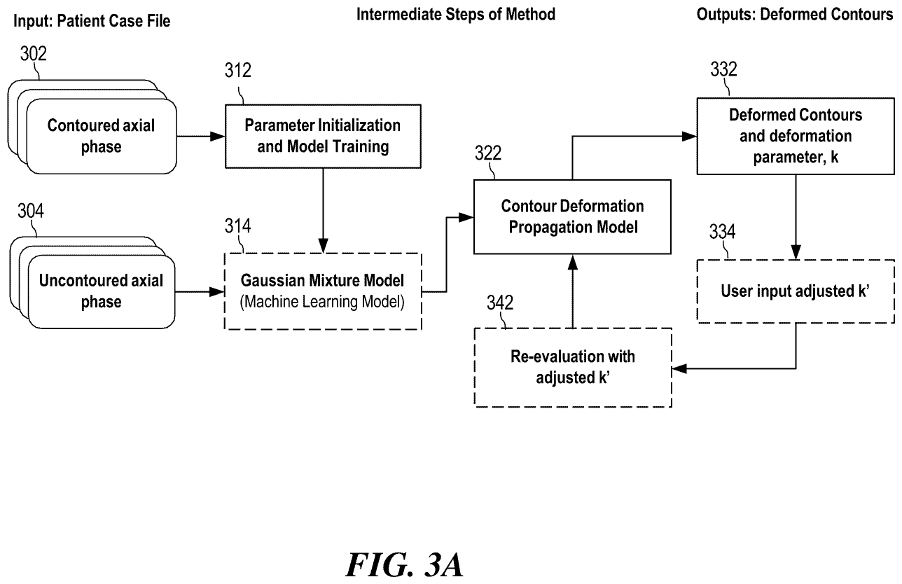

In one example, the method may include a generalized algorithm, wherein a first step in a generalized algorithm, as shown in FIGS. 3A and 3B, includes processing data from a case file and delineating ROI. Each patient case file may include axial slices from a set of medical scan images over time of a physiological cycle that causes involuntary movement of the internal systems, and the contours drawn by a dosimetrist 302,352. The generalized algorithm determines a transform function that accounts for the difference between a fixed and moving image, such as an image captures during a physiological phase (e.g. breathing phases) using a contour deformation propagation model 322.

The generalized algorithm includes a Gaussian Mixture Model, which is combined with parameter initialization and training-prediction implementation 312. First, initial parameters are derived from number histograms of the medical image, in which manually or automatically selected peaks in the distribution represented the means of the clusters. User-initialized cluster parameters are used in the automated peak selection and the corresponding calculation of the initial cluster parameters for the exemplary method, specifically for the GMM. The number of peaks in the histogram represents the number of clusters with which the model is allowed to categorize the voxels. The standard deviation for each cluster is calculated as half of the difference between the mean of the cluster for which the standard deviation is desired and the nearest mean of a different cluster. With this parameter, the weights for each cluster (which represents the expected proportion of voxels that will be allocated to the cluster; may also be thought of as the size of the homogenous region) is calculated by numerically integrating the region. Numerical integration is carried out by multiplying the bin widths by the height (the density) of the corresponding bar of the histogram over a range of HU numbers that are plus and minus one standard deviation away from the corresponding mean of the cluster for which the weight parameter is desired.

In this method, the order and selections of training versus prediction data is leveraged to optimize prediction performance of the GMM 314. The method, including initialized cluster parameters and use of the corresponding segmented images for deformation field calculation and contour propagation, yields a multitude of use cases under the same machine learning framework. The first involved training the GMM through iteratively refining the cluster parameters to be representative of an initial time image (or an averaged time image). Each iteration involved calculating the probability that each voxel belongs to each cluster, and the maximum probability for each voxel determined which cluster that voxel was assigned to when calculating the mean, variance, and weight for each cluster for the next iteration.

The machine learning framework allows multiple use cases, including sub-organ level segmentation and fast deformation tracking. This involves first defining a region of the axial scan that represents only the ROI using image morphology. The parameter initialization is then carried out only on the voxels within this region, and the subsequent segmentation from the GMM 314 is delineated between the healthy tissue and cancerous tissue within the lung itself. In total, the method provides a mathematically sound and computationally inexpensive solution for deformable image registration and provides the user with a tunable factor to scale the contour diffusion. This tunability provides robustness to an output that requires physicians to interact with the output to adjust to other factors such as poor image quality, which may alter the reliability of the output.

Deformation parameters are categorized by utilizing three separate models. Each set of deformation parameters are paired with a performance metric, while setting the primary optimization criteria (e.g. minimizing incident doses to organs at risk (OARs) and/or consistent target region location and shape).

The generalization algorithm initializes the deformation parameters by extracting the mean and variance for each “cluster” that the GMM classified voxels into by utilizing the contour information stored in the patient case file (e.g. manually drawn contours on the images), hence capturing a higher degree of individualization based on the patient's specific organ movement. Additionally, the algorithm initiates “secondary” clusters which account for the tissue that may not belong to a drawn contour and account for the inhomogeneity of contoured regions. By accounting for inhomogeneity, the method can enact sub-organ level segmentation, which further assists in sparing healthy tissue of any delivered dose. Lastly, because the GMM is categorizing the voxel HU values into one of many predefined Gaussian distributions, the statistical nature of the algorithm accounts for variability in attenuation when capturing the scans and the limited resolution that may result in one region being represented by a single voxel in the first axial slice and being distributed among multiple voxels in the next axial slice. Although studies in the past explored the capabilities of GMM for simple image segmentation for auto-contouring, the exemplary method initializes cluster parameters and optimizes the operations performed on the results of the GMM. By intimately tying the clusters to both the contours, the background tissue, and the expected inhomogeneity of contoured regions, the exemplary method provides patient-specific treatment planning while providing a more streamlined approach. Additionally, the exemplary method uses the segmented image for diagnostic and/or treatment planning rather than the segmented image being the final goal or deliverable of the algorithm.

After the image is segmented by the GMM into the different ROIs, the deformation field is obtained to propagate the already delineated contours across all the different physiological phases using the contour deformation propagation model 322. This output from the GMM serves to improve the fidelity of the segmentation by selectively partitioning different areas, which inherently diminishes noise artifacts. Previous deformable image registration (DIR) methods use the global information of the image to get a global deformation field and then push the contour in the desired direction based on a transformation applied to the fixed image.

Here, the disclosed generalization algorithm uses only the information from the ROIs and the already segmented image from the GMM results. Using a non-trivial diffusion equation (i.e. the diffusion coefficient is not uniform) to find the deformation field. In some aspects, a finite element analysis capable of processing linear and nonlinear systems is used.

In one embodiment, a modified Poisson's equation is used to is state the problem as follows:

Ε·(Γ(x, y, z)∇Φ(x, y, z))=S(x, y, z)

Given a fixed F_{GMM} and moving M_{GMM} image already segmented by the GMM, and a set of closed contours ∂C={∂_{C1}, ∂_{C2}, . . . , ∂_{C}N}, the diffusion coefficient Γ(x, y, z) and source term S(x, y, z) for the Poisson equation are as follows:

$$S ( x , y , z ) = \{ \begin{matrix}1 & if p ∈ int ( \partial C ) + \partial C \\ 0 & otherwise\end{matrix}$$

In this way, a flux representing the deformation field is determined using the source and the diffusion coefficient. That is:

T: p→p+T(p)

Where T(p) is defined via a displacement function based on the gradient of the field,

T(·)= [symbol] (·, k∇Φ)

Formulating the source to exist only in the regions of interest (i.e. OAR, PTV, etc.) allows those regions defined in the fixed image to diffuse to the moving image. The diffusion coefficient when (F_{GMM}−M_{GMM})≠0 will be inversely proportional to the difference between the original fixed and moving image, therefore, zones with large differences will have a small diffusion coefficient making it difficult for the source to diffuse through those zones and not to diffuse if the diffusion coefficient is large.

A displacement field is determined from [symbol] (·, k∇Φ) and is applied to the image. In this way, this field is proportional to the gradient field, which contains the magnitude and direction of how the source/s are diffusing. Then, the optimization problem is reduced to obtaining a single parameter, k, which provides significant computational resources optimization compared to conventional methods. This exemplary method relies on mathematical derivations which assume smooth and small displacements of delineated regions across different physiological phases, such as for 4D CT scans.

In the exemplary method, as shown in FIG. 3A, the deformed contours are output for visual inspection together with the deformation parameter, k, 322. In some aspects of the method, the user provides an adjusted k′ 334, then the contour deformation propagation model is re-evaluated with the adjusted k′ 342.

In another embodiment, a Navier-Stokes equation tailored for fluid-like tissue behavior, such as a very dense fluid. A region may have a certain fluid profile, which produces the original contour of the tumor, and the fluid profile may change thus producing a deformed image.

In yet another embodiment, a Lagrangian mechanics-based algorithm may be used that accounts for energy conservation to obtain the deformation field.

In other embodiments, an elastodynamic equation for capturing time-dependent deformations may be used. For example, in a system where the tumor is a deformable solid, having properties similar to a rubber, and obtaining the displacement field by applying a force vector and getting the stress tensor of the body by relating the difference in medical image units between two phases.

In other aspects of the method, as shown in FIG. 3B, the contour deformation propagation model 372 provides the deformed contours for display output 382 and to a second machine learning model 392. The second machine learning model 392 functions to model dose distribution across all the physiological phases and provide dose bounds for the current radiotherapy plan 394.

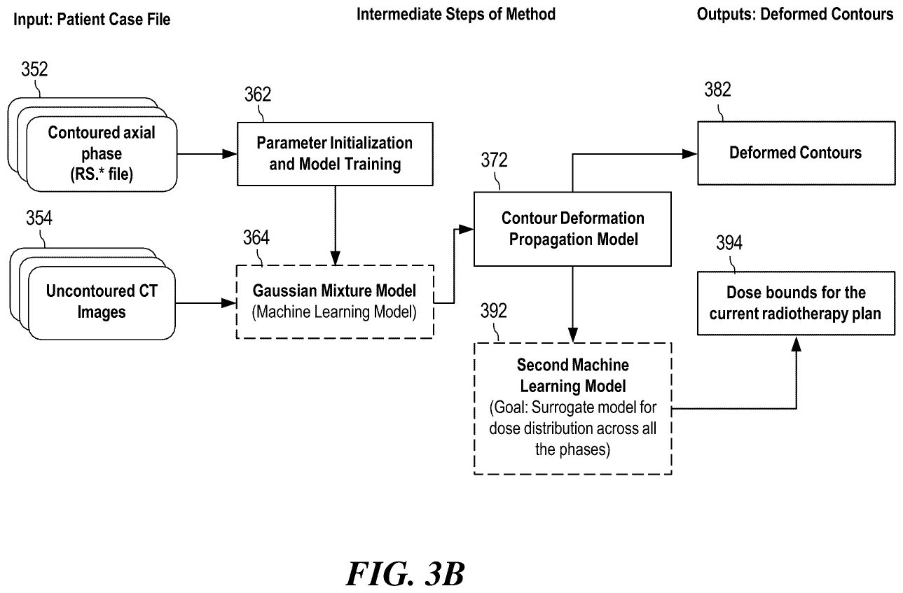

FIG. 4 shows an illustrative embodiment of an exemplary system. In some aspects, the exemplary system 400 for deformable image registration includes: two or more sets of medical scan images, a user device 430 including a means for input and output; and one or more computing systems 400 including one or more processors 404 and one or more storage devices 410, wherein the one or more storage devices have instructions stored thereon, that when executed by the one or more processors, causes the one or more processors to perform a method 412 (i.e. the deformable image registration module), the method including: receiving two or more sets of medical scan images for a patient including a first 432 and a second 434 set, wherein the first set of medical scan images 432 includes pre-drawn contours; delineating (e.g. clustering) one or more ROI in the second set using a trained clustering machine learning algorithm; determining deformation contours for the one or more regions of interest (e.g. tumor) in the second set using a finite element analysis algorithm over one or more of a plurality of phases of physiological cycles (e.g. phases of breath cycle); and outputting the deformation contours relative to the one or more ROI; wherein the deformation contours are subsequently used for diagnostic and/or treatment planning.

Wherein, in some aspects, determining deformed contours for the second set includes: deriving a deformation field using the finite element analysis algorithm, wherein the deformation field is dependent on an adjustable parameter (i.e. k); applying the deformation field over the delineated one or more regions of interest over one or more of a plurality of phases of physiological cycles thereby forming deformed contours.

In some aspects, the system is configured to receive an adjusted value of the adjustable parameter from a user, wherein in response to the user adjusting the adjustable parameter, the deformed contours are re-evaluated with the adjusted parameter (e.g. k′) and the re-evaluated deformed contours are output.

### Example 1: Using Gaussian Mixture Models and Modified Poisson's Equation as Deformable Image Registration for Non-Small Cell Lung Cancer

For Non-Small Cell Lung Cancer (NSCLC) patients, the respiratory-induced motion of abdominal tissues poses a significant hurdle to effective irradiation. Hence, it was crucial to implement deformable image registration (DIR) methods informed by 4-dimensional computed tomography (4D-CT) data, that were both time-efficient and accurate. The ultimate objective was harmonizing the dynamically changing anatomical features with clinically viable dose delivery, including target definition, gross tumor target volume (GTV) tracking, organ-at-risk (OAR) conservation, and respiratory gating. Traditional DIR approaches, such as Horn-Schunk optical flow [1], DEMONS [2], and B-spline-based algorithms [3], often falter in efficiently handling voluminous 4D-CT datasets [4]. As a result, the disclosed exemplary method provides optimized DIR techniques tailored to the specific challenges of stereotactic body radiotherapy (SBRT) in NSCLC treatment planning.

The exemplary method integrated predictive models of tumor segmentation, deformation, and medical data into fast, reliable, robust, and efficient solutions to deliver optimal treatment to each patient.

In this context, the exemplary method and system provide an algorithm that combines machine learning and a modified Poisson equation to propagate GTV contours across the different respiratory phases in a 4D-CT scan in an efficient runtime and accurate manner when juxtaposed through similarity metrics with current implemented methodologies. The exemplary method first segmented the outer body from the bed and surrounding air in the CT scan; then, in this segmented domain, a GMM was run to cluster the different structures inside the lungs (tissue, airways, tumor masses, or nodules, vessels, lung parenchyma, etc.) along with OARs. Using the previous GMM segmentation, a variable diffusion field for the Poisson equation was created, and the discretized modified Poisson's equation was solved over the entire ROI to get a diffusion field. The gradient of this diffusion field was proportional to the deformation field, and fine-tuning provided a robust registration. To validate the computational framework, a benchmark of the contours generated from the combined GMM-Poisson workflow against contours generated from DEMONS DIR for analogous propagated contours is presented.

### Materials and Methods

Patient Cohort. The Emory Proton Therapy Center provided six anonymous cases of patients diagnosed with NSCLC. Chest CT scans were performed on all patients in the headfirst-supine position. In addition, each patient underwent a 4D-CT scan under controlled breathing, utilizing a SIEMENS tomograph with a Br40s reconstruction kernel. The respiratory motion management technique used during the 4D-CT scan was free breathing. The 4D-CT images were reconstructed into ten respiratory phases, and the body was sliced into 311 slices, each 1.5 mm thick, for each phase. Each slice was divided into 512 rows and 512 columns, with an isotropic pixel spacing of 0.977 mm. The acquisition parameters included a peak kilo voltage output of 120 kV and an X-ray tube current of 75 mA. Moving forward in the work, each “Case” (or i.e., “sets of medical image scans”) to a set of 4D-CT scans of a single patient.

Algorithm Overview and Workflows. The algorithm of the exemplary method includes executing a series of mathematical operations and transformations, beginning with inputting a 4D-CT scan. Specifically, the algorithm includes evaluating a function, fᵢ(x, y, z), to represent the i-th phase of the respiratory cycle, where (x, y, z)∈Ω. Here, Ω denotes the entire physical space associated with the 512×512×311 voxels pertinent to a single phase of a 4D-CT scan.

The first step in the algorithm involved isolating the body within the CT scan, resulting in a mask that defined the location of the outer perimeter of the body. This step yielded a subset of physical space, denoted as Ω_{body}⊂Ω. Once this segmentation was obtained, the region, Ω_{body}, defined the domain for the GMM to be executed on and minimized the number of voxels that must were iteratively clustered. The GMM created a set of clusters defining organs and other internal structures, including some sub-organ level structures within the lungs. That means that given a fᵢ(x, y, z) with (x, y, z)∈Ω_{body} was transformed into a f_{GMM}(x, y, z)=j if (x, y, z)∈Ωⱼ where j=1, . . . , N represent the different clusters obtained after running the GMM segmentation and Ωⱼ⊂Ω_{body}∀j. This transformation can be represented in eq. 1.

$${T}_{GMM} : {f}_{i} ( x , y , z ) {⟶}^{GMM} {f}_{GMM} ( x , y , z ) ( x , y , z ) ∈ {Ω}_{lung} \qquad ( eq . 1 )$$

Based on the cluster information obtained from f_{GMM}, a field for the diffusion coefficient or diffusivity, denoted by Γ(x, y, z) was constructed; this field was used in a modified Poisson equation with a fixed source. The underlying principle behind this definition of diffusivity is as follows: suppose we consider some quantity ϕ(x, y, z) that diffuses, which could be a chemical concentration in a dilute solution or a temperature field in a heat-conducting system. If the change between a fixed and a moving image (e.g., two different respiratory phases in our 4D-CT scan) is inversely proportional to the local diffusivity, then the flux Φ=∇ϕ(x, y, z) is proportional to the deformation field. The physical interpretation of this relationship is that where there is a difference between two images, the diffusivity is small, and it is significant if there is no difference between them. As a result, the quantity ϕ will tend to flow towards regions with higher diffusivity in the steady-state solution. Therefore, the field ∇ϕ becomes proportional to the deformation field, which indicates how to push the GTV contours from one phase to another.

The calculation of the gradient field allowed for the application of a transformation field belonged to the GTV, denoted by (x, y, z)∈Ω_{GTV}, wherein a medical professional delineated these points. The warping transformation process employed is illustrated in eq. 2. Notably, these transformations do not preserve volume nor topological properties, which is acceptable given that the tumor's shape may change over time.

$$( x , y , z ) {⟶}^{T ( \cdot )} ( u , v , w ) ∀ ( x , y , z ) ∈ {Ω}_{GTV} \qquad ( eq . 2 )$$

$$T ( x , y , z ) = ( x + {p}_{x} , y + {p}_{y} , z + {p}_{z} ) = ( u , v , w ) \qquad ( eq . 3 )$$

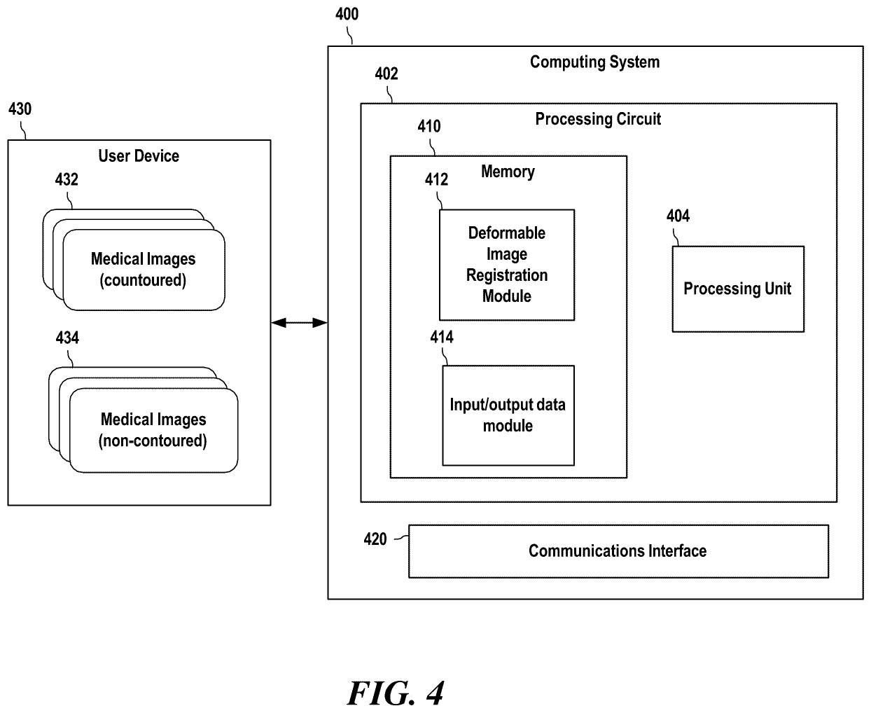

Furthermore, (pₓ, p_{y}, p_{z})=kΦ=k∇ϕ, where k is a proportionality constant. A summary of the algorithm can be seen in FIG. 5.

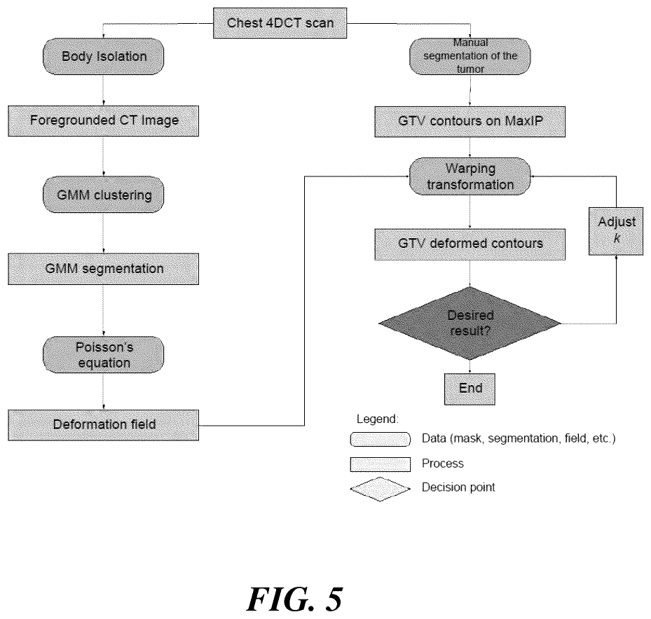

The Gaussian Mixture Model. The GMM is an unsupervised machine learning clustering algorithm that leveraged initialized parameters to categorize (i.e., cluster) input data points into one of many possible clusters. The GMM operates on the base assumption that each data point was randomly sampled from one of a predetermined number (K) of Gaussian probability distribution functions (PDF) with unknown parameters (i.e., mean, standard deviation, and weight). The number of probability distributions (K) may be determined from the a single image, wherein the number is chosen as the number of peaks in an image or related signature (e.g., histogram of HU number from CT, FDG avidity in PET, MRI sequences such as T₁/T₂ weight imaging, diffusion restriction, MR spectroscopy). The initial parameters of each K—mean, standard deviation, and weight—can be randomly or pseudo-randomly generated or estimated by some other means. For example, FIG. 6, top panels, show a medical image and related initial peak selection with randomly generated parameters.

An individual Gaussian distribution to which a data point may belong is called a “cluster,” where the k^{th} cluster's PDF is given by the normal distribution with a mean μ and a σ standard deviation. The term “mixture” stems from the assumed formulation of a probability distribution function that describes the entire data set (p(x)) as the mix of the K

Gaussian distributions were defined as follows:

$$\begin{matrix}p ( x ) = {\sum }_{k = 1}^{K} {π}_{k} ( ( \frac{1}{{σ}_{k} \sqrt{2 π}} exp ( \frac{- {( x - {μ}_{k} )}^{2}}{2 {σ}_{k}^{2}} ) ) ) & ( eq . 4 ) \\ = {\sum }_{k = 1}^{K} {π}_{k} 𝒩 ( x \vert {μ}_{k} , {σ}_{k} ) & ( eq . 5 )\end{matrix}$$

The term πₖ is the weight of the k^{th} cluster and is sometimes referred to as the “mixing coefficient”. The weight of each cluster represents the fraction of data points that are expected to be allocated to that cluster such that it adds up to one.

Regarding the available data set, 4D-CT scans, a single CT image was considered a univariate collection of observations; each voxel was a single observation assumed to belong to one of many possible body substructures, each substructure being a cluster. The single feature that defined an observation (i.e., a voxel) is the Hounsfield Unit (HU), which is a water-based normalized linear attenuation coefficient for a photon passing through the body [26]. Within the framework of the GMM, it was assumed that a given substructure of the whole body can be represented as a Gaussian distribution with a mean value associated with the expected radio density of the structure and standard deviation arising from multiple noise sources.

The training/fitting of a GMM entailed maximizing the likelihood that a mix of Gaussian distributions would give rise to the observable data. As such, the probability that a given data point, xᵢ, was generated from cluster k must be calculated for N data points for every cluster, which produces an N×K matrix where each row corresponds to a data point and each column corresponds to the probability that the data point was generated from the cluster represented by that column. In a Bayesian formulation, and individual entry into the N×K matrix, rᵢₖ, is simply the posterior distribution and is given by:

$${r}_{ik} = \frac{{π}_{k} 𝒩 ( {x}_{i} \vert {μ}_{k} , {σ}_{k} )}{{\sum }_{j = 1}^{K} {π}_{j} 𝒩 ( {x}_{j} \vert {μ}_{j} , {σ}_{j} )} \qquad ( eq . 6 )$$

The numerator of eq. 6 represented the absolute probability that the HU value of a given voxel, xᵢ, was a result of that voxel belonging to a lung substructure represented by the Gaussian distribution, which took into account the expected radio-density of the structure, μₖ, the influence of noise in the CT scan on the substructure, σₖ, and the size of the substructure relative to other present substructures or air, πₖ. The denominator of eq. 6 served to normalize the probabilities such that summing across a row in the N×K matrix resulted in a value of 1, meaning the voxel must belong to one of the K clusters. This calculation then determines the shape of the clusters for the subsequent iteration and each iteration's “quality” to maximize the expectation that the set of Gaussian distributions was a logarithmic likelihood function that determines the correct posterior Gaussian mixture. Ten training/fitting iterations were implemented to ensure convergence of the log-likelihood. In FIG. 6, lower panels, the set of trained Gaussian distributions and the related initial segmentation are shown.

Conceptually, the GMM served to discretize the domain Ω_{body} such that the influence of the many sources of noise (i.e., electrical, quantum, etc.) in a CT scan were entirely encompassed by the standard deviation fitted to a given cluster. This discretization allowed for the next step of the DIR process, the modified Poisson equation, to be less affected by infinitesimal variations of HU values of voxels that belonged to the same substructure.

As a form of unsupervised learning, the GMM was trained on the average image from the ten respiratory phases. Training on an averaged image allowed the patient to serve as their own training data without training on a cohort of test data. Once the model was trained on the averaged image, the K Gaussian distribution parameters were used to categorize every voxel for the ten respiratory cycle phases. As the set of learnable parameters was used to cluster the CT image for a particular time phase, the time phase was incorporated into the training for updating the set of PDF parameters, mimicking an additional iteration of training.

The most challenging portion of implementing a successful GMM was initiating starting points for the distributions' means, standard deviations, and weights. In order to maximize the automation and utility of the algorithm, pseudo-random distributions were generated. The set of cluster means was initialized as evenly spaced values between the minimum and maximum HU values for the current averaged slice. Moreover, the corresponding weights were uniform, assuming no prior information about the relative sizes of the clustered structures.

Poisson's equation. The utilization of a modified Poisson's equation for deriving a deformation field was based on interpretation of the deformation process as a stationary, non-linear diffusion phenomenon. Qualitatively, this was likened to a scenario where heat (or some analogous property, denoted as ϕ) diffuses through a medium. However, unlike classic diffusion processes, where the diffusion coefficient Γ was constant, it varied spatially, i.e., Γ(x, y, z). Quantitatively, this spatially varying Γ was formulated to depend on the differences between the two images being compared. Hence, diffusion's “easiness” or “difficulty” corresponded to the local similarity or dissimilarity between the two images.

The diffusion coefficient Γ thus became a function of space Γ(x, y, z), making the process analogous to a stationary case of a non-linear diffusion equation. This formulation of Γ was done to capture the variability arising from the differences between the two images. A novel method was proposed that employed information from the GMM clustered images to define this non-uniform diffusion field. The method also leveraged a particular ROI to accelerate the solution.

Mathematically, the modified Poisson equation representing this steady-state non-linear diffusion process is:

Ε·Γ(x, y, z)∇ϕ(x, y, z)=S(x, y, z) (eq. 7)

In this equation, S(x, y, z) serves as the source term, which will be chosen to be uniform and its magnitude equal to one. ϕ(x, y, z) represents the gradient of the property ϕ attempting to diffuse through the medium.

Therefore, given a fixed F and moving M images that are segmented by the GMM process (F_{GMM} and M_{GMM} in eq. 8), and a set of closed contours ∂C={∂_{c1}, ∂_{c2}, . . . , ∂_{cN}} drawn by a specialized physician, the diffusion coefficient Γ(x, y, z) for the Poisson equation can be formulated as depicted in eq. 8.

$$Γ ( x , y , z ) = \{ \begin{matrix}{a ( F ( x , y , z ) - M ( x , y , z ) )}^{- b} & if ( {F}_{GMM} - {M}_{GMM} ) < 0 \\ {Γ}_{max} & otherwise\end{matrix} \qquad ( eq . 8 )$$

The motivation behind this formulation was to generate a flux representative of the deformation field, defined as T: p→p+T(p). The transformation field T(p) was itself calculated via a displacement function [symbol] based on the gradient of the field k∇Φ.

T(·)= [symbol] (·, k∇Φ) (9)

The key to diffusion coefficient modeling lies in their spatial variability; this formulation analyzes how the field, supplied by the uniform source, diffuses from the fixed image to the moving image. In this regard, the diffusion coefficient Γ was formulated to hold large values in regions where there was no discernible difference between the clustered fixed image (F_{GMM}) and the clustered moving image (M_{GMM}). In contrast, a reduced value of Γ was attributed in areas where a discrepancy exists. Consequently, the diffusion coefficient is built to be inversely correlated with the differences between the original fixed and moving images. This formulation implies that zones characterized by significant differences will be associated with a small diffusion coefficient, complicating the source's diffusion through these regions and vice versa when the diffusion coefficient was significant.

The displacement function [symbol] (·, k∇Φ) yielded a displacement field proportional to the gradient field, which in turn influenced the magnitude and direction of the source's diffusion. The optimization challenge, therefore, was narrowed down to the determination of a singular parameter k, which was fine-tuned to produce optimal results. Initial estimations used DEMONS registration as a baseline and subsequent adjustments based on visual evaluations. This approach added robustness to the function that governs the propagation of the contour within the respiratory phases. This example implemented a diffeomorphic transformation that was prevalently employed in medical image registration [27]. This function accepted the displacement field as an input and sequentially applied the corresponding transformation from voxel to voxel. The initiation of this process necessitated the identification of the interior points of the contours, expressed as ∂C=∂_{c1}, ∂_{c2}, . . . , ∂_{cN}, and the subsequent construction of a solid object encapsulating the GTV or any ROI intended for propagation. The transformation delineated in eq. 9 was applied to each constituent point p of the solid ROI upon crafting these solid objects.

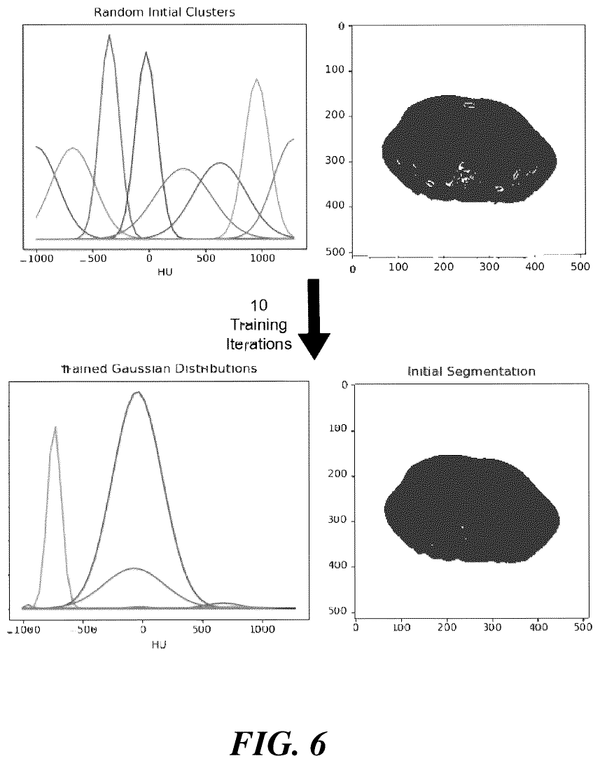

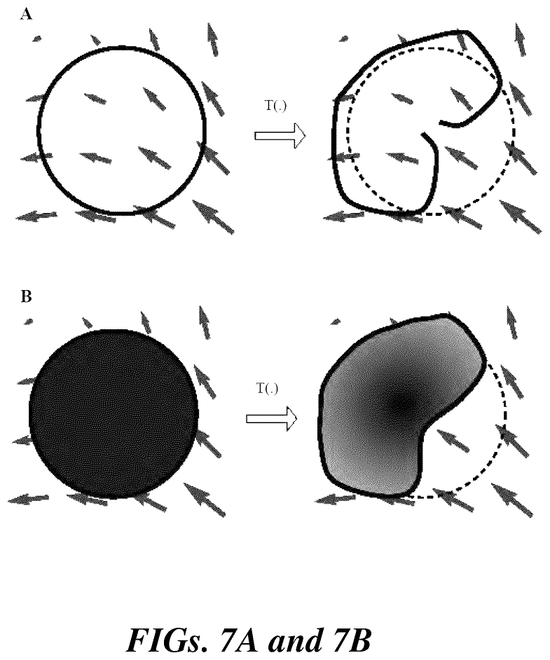

There are two foundational considerations in this methodology. First, direct application of the transformation to the contour points was culminated in a non-closed deformed contour, owing to the potential superposition of two points (FIG. 7A). To avoid this superposition, the transformation was initiated with a set encompassing all interior points of the contour, as visualized in FIG. 7B. Second, the algorithm commenced with a voxelized image but transitioned to a continuous coordinate space during the deformation field application. Thus, a reconversion to the voxelized discrete space was required once the transformation was complete.

To achieve this reconversion, a conservative approach was adopted. It involved dividing the continuous space into the original voxelized space derived from the CT scan. Here, a continuous value ranging from zero to one was assigned, predicated on the field's average value in one voxel, as depicted by eq. 10. In most interior voxels, this value will equal one, but boundary values will be less than one due to the warping function applied. Subsequently, in the discrete space, the final deformed contours can be extracted by identifying the boundaries of the set, using a well-established boundary-detection algorithm such as the canny edge detector algorithm. These two steps were implemented using the Simple-ITK [28] python library [25].

$${ϕ}_{i} = \frac{{\int }_{{Ω}_{Voxel}} ϕ ( x , y , z ) dV}{{\int }_{{Ω}_{Voxel}} dV} \qquad ( eq . 10 )$$

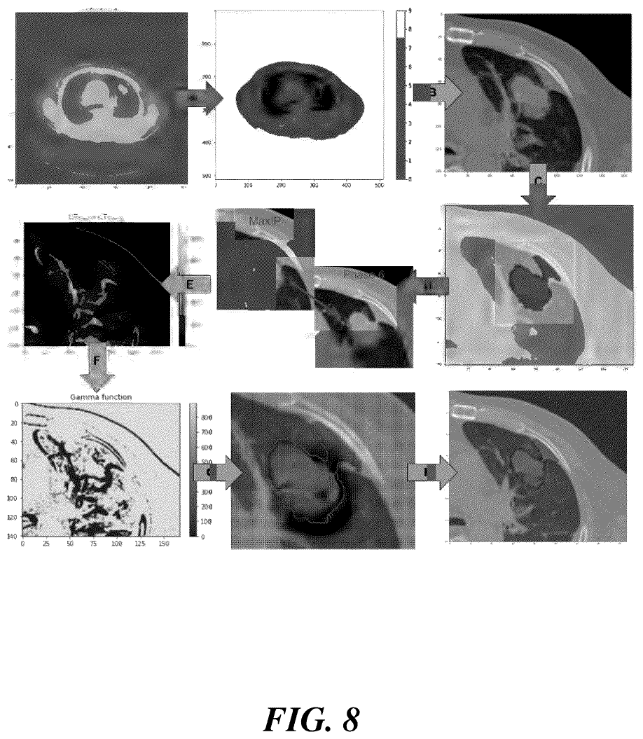

Overall, FIG. 8 illustrates the methodology for propagating the contours.

### Results

GMM+Poisson compared to DEMONS registration method. The initial benchmark to assess the presented algorithm involves optimizing the proportionality constant k that multiplies the deformation field k∇Φ. This process aimed to evaluate our proposed method's performance compared to the Fast Symmetric Forces DEMONS implementation in ITK [29], an extensively tested algorithm.

In implementing the DEMONS algorithm, 100 iterations were employed for a smooth displacement field and a standard deviation equal to 2.0. For the example implementation of the GMM combined with Poisson's equation, the body was segmented into eight clusters, and GMM segmentation converged in ten iterations. Concerning the Poisson equation, through rigorous trials, the optimal parameters for constructing the diffusion field Γ(x, y, z) from eq. 8 were identified as a=1×10⁻⁵ and b=1.5. The CT scans were also normalized between 0 and 1 to generate the diffusion coefficient.

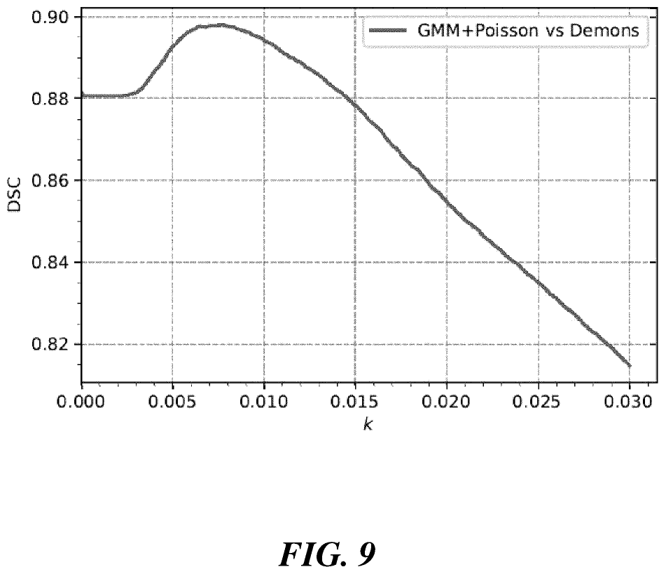

The optimization process ultimately determined that the value of k most closely aligned with the DEMONS results was k=0.0075. This process entailed scanning a range of k values, from 0 (indicating no deformation) to 0.03, and computing the Dice Similarity Coefficient (DSC) against the results from DEMONS for each. FIG. 9 illustrates the evolution of the DSC as the proportionality constant k was varied, shedding light on the intricate relationship between deformation and image alignment.

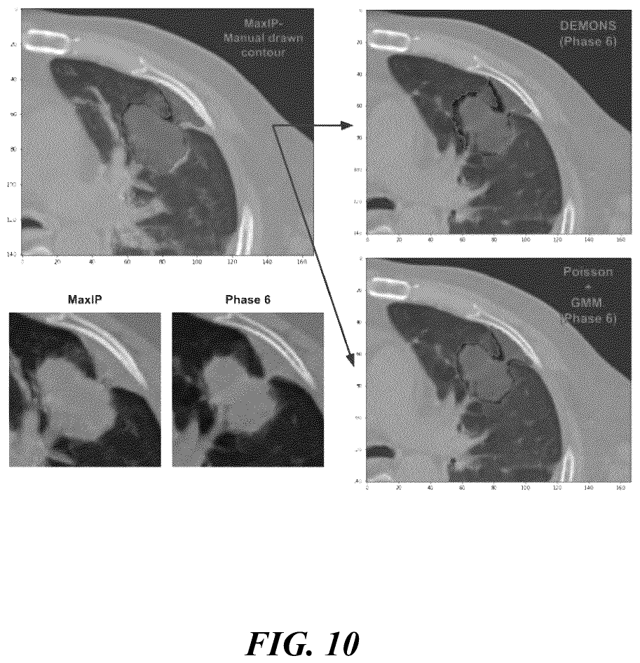

FIG. 10 visually compares the exemplary algorithm with the conventional, DEMONS, algorithm, where one axial slice of one patient is displayed. A specialized physician drew the contours, and for visualization purposes, contours being deformed to phase 6, which was found to be the most different from the manually drawn contours, are displayed. From FIG. 10, it is evident that the developed algorithm matched the performance of the conventional medical imaging registration method, such as DEMONS [30 ] [31]. The consistency of results between the algorithms emphasizes the potential applicability of the method in advanced medical imaging contexts.

Algorithm robustness on k. A distinctive feature of this algorithm is the ability to modify the final contour after calculating the deformation field, offering robust adaptability as it allows for slight proportional modifications to the final contours on the fly. Six different values of k were tested to gauge their effect on the deformation field k∇Φ. These selected values were chosen to display noticeable deformation in most cases. However, the selected values of k lack clinical relevance; they were chosen to illustrate variations even to an untrained eye. A practicing physician can finely tune the value of k to better conform to the GTV, achieving greater precision in contouring. A reference range is provided here just for guidance.

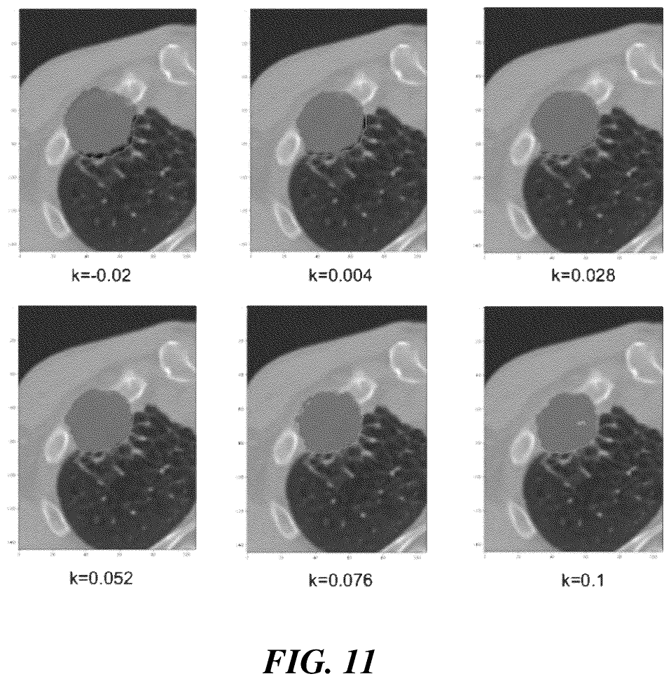

FIG. 11 showcases one patient where the GTV is embedded within the lung wall. This complexity rendered the DEMONS registration of the tumor across different respiratory phases more challenging. Nonetheless, the presented algorithm outperformed DEMONS. In addition, FIG. 11 demonstrates the algorithm's was sensitivity to variations in k, underscoring the robustness and adaptability of the method for diverse clinical scenarios.

A comparison between GMM+Poisson and DEMONS DIR methods. Another visual assessment conducted to evaluate the performance of the exemplary algorithm involved a comparison against DEMONS DIR. This comparison was performed across five of the six patients (i.e., cases) in the patient cohort, with one case excluded due to the availability of only one registered respiratory phase.

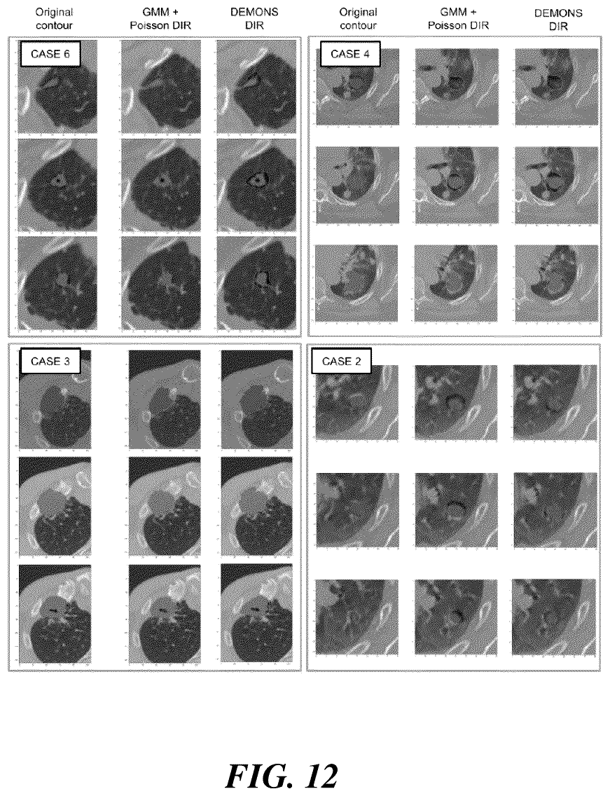

To scrutinize both registration methods, the respiratory phase that deviated most from the manually drawn contour was selected, and the registration was carried out between this projection and that point in time. FIG. 12 displays the differential performance across a series of chosen slices. These selected slices were strategically set to cover the tumor's upper third, middle, and lower third. The upper third means the part closer to the head, providing a comprehensive visual analysis.

In FIG. 12, Case 6 illustrates where the exemplary method surpassed the conventional, DEMONS, registration. In the DEMONS method, the contours were incorrectly propagated over healthy tissue, whereas the exemplary registration technique finely tuned to follow the tumor across all the slices accurately. Case 4 of FIG. 12 presents a more complex registration scenario, with both algorithms exhibiting imperfections. In the upper third of the tumor, both algorithms excluded what appears to be healthy tissue and exhibited similar performance. However, in the middle section of the GTV, DEMONS DIR mistakenly included more healthy tissue, although neither algorithm precisely captured the deformation. In the lower third, both methods failed to encompass the whole GTV, with DEMONS additionally including some healthy tissue. Overall, GMM+Poisson exhibited superior visual performance in this case toward capturing the GTV, notwithstanding some minor drawbacks.

In Case 3 of FIG. 12, both algorithms demonstrated adeptness in capturing the tumor between the manually drawn contour and the respiratory phase. However, the robustness of GMM+Poisson allowed for finer GTV contour adjustments to minimize the inclusion of healthy tissue. DEMONS' registration misidentified some of the lung parenchyma and chest regions in the lower and upper thirds of the tumor. However, it better captured the GTV near the bony structures in the tumor's middle section.

Lastly, Case 2 of FIG. 12 shows where DEMONS outperformed the registration method presented herein. Here, the GMM+Poisson algorithm seemed biased towards including more healthy tissue than warranted whereas missing parts of the GTV. Conversely, DEMONS DIR captured the GTV across the entire tumor.

A second comparative study presents further differences between the GMM+Poisson DIR versus the DEMONS for every respiratory phase. Both registration methods were carried out on all phases in Case 1 of FIG. 12, and the DSC was calculated between the GTVs resulting from either method. Although in some scenarios, the GMM+Poisson method can fulfill the same role as DEMONS. The exemplary method provides additional utility over the conventional methods, such as providing additional functionality to clinicians to quantify how, when, and where the exemplary method deviates or aligns with existing ones (i.e. DEMONS).

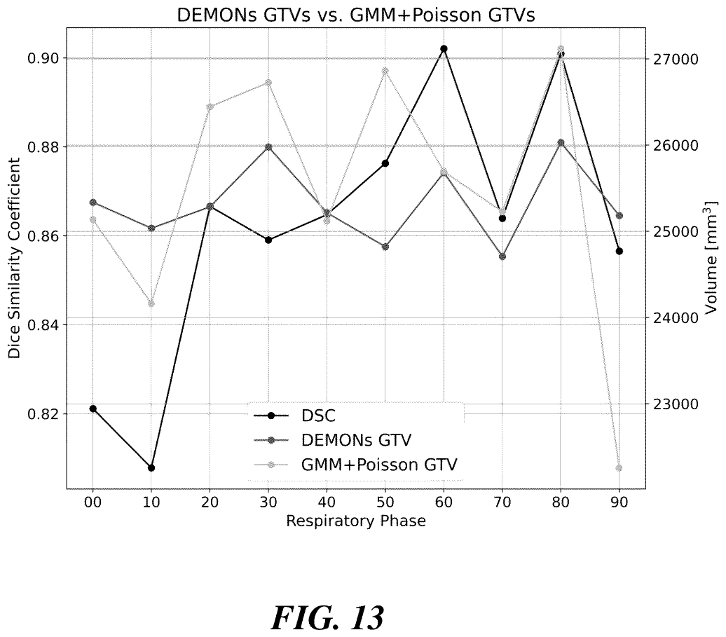

As illustrated in FIG. 13, the method's utility is evident through the volume metrics and DSC analysis during various phases of respiratory motion. The data indicates that our GMM+Poisson algorithm effectively captures the geometric details of the GTVs, maintaining a substantial overlap with the benchmark DEMONS GTVs across different respiratory phases. Notably, while there are instances where the GMM+Poisson GTVs diverge from the DEMONS baseline—reflected in lower DSC values—this typically coincides with a reduced volume of the propagated contour. This phenomenon merits further investigation to determine the clinical significance, as the decreased volume has the potential to enhance OAR sparing without compromising tumor coverage. Moreover, the GMM+Poisson method exhibits adaptability, with its volume changes across respiratory phases potentially offering a more accurate representation of the dynamic tumor environment. This feature could be critical in capturing the true extent of respiratory-induced tumor motion, which may not be fully represented by more static models like DEMONS. While further dosimetric validation is necessary, the initial findings suggest that with expert clinical validation, this technology may refine tumor tracking during radiation therapy, improving treatment precision and patient outcomes.

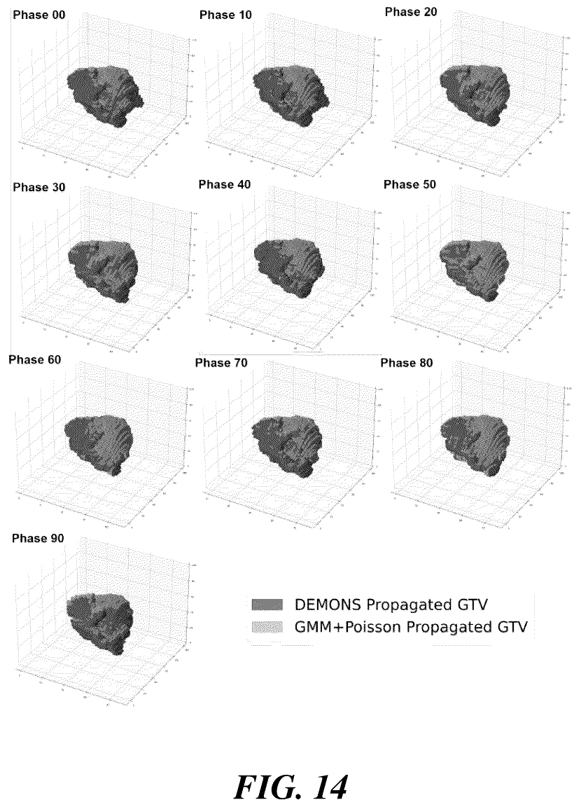

FIG. 14 shows a qualitative analog of the comparative study. As expected from the analysis of FIG. 13, and from the view given by the three-dimensional renderings of the GTVs, it seems that the phases with relatively low DSCs (Phases 00 and 90) tend to have smaller volumes predicted by the GMM+Poisson method. Conversely, Phases 60 and 80, which have the highest DSCs, see the GMM+Poisson method generate GTVs that encompass more than the DEMONS counterparts. Thus, the GMM+Poisson method seems to amplify the tendency of the DEMONS DIR, whether it is a lesser or greater volume. This amplification may indicate the GMM+Poisson method capturing more dramatic changes in the size and shape of the GTV due to respiratory uncertainties.

### Discussion

In this study, a deformable image registration method was introduced and empirically validated on NSCLC patients, which fulfilled an existing need in the field of medical imaging for NSCLC. Unlike previous attempts to benchmark available registration algorithms for this particular type of cancer [32], [33], the exemplary system and method was adapted for the unique challenges posed by NSCLC. The implementations noted in Fabri's work [34], restricted to phantoms, lacked the complexity, random motion, and noise inherent to actual CT scans of human subjects, limiting their applicability.

The exemplary method leveraged the distinctive morphological characteristics of NSCLC tumors, capitalizing on their common location inside or near the lungs. Based on the lungs' air content as a distinguishing feature, the GMM was employed to facilitate better registration. Building upon this conceptual framework, a numerical solution to the Poisson equation was devised with a variable diffusion coefficient in a 3D geometry. In contrast to previous attempts, such as that in Mohanalin's work [35], which utilized a constant diffusion field in a 2D setting, the exemplary method incorporated critical information regarding organ structures via the GMM, enhancing the fidelity of the diffusion field construction.

Regarding time complexity, the exemplary method was comparable to that of DEMONS registration, as evidenced by empirical timings gathered during testing, such as an 18-second duration for solving the Poisson equation on a mesh sized (167×141×68), which corresponds to the ROI size, compared to approximately 15 seconds for DEMONS.

Furthermore, the exemplary method enabled on-the-fly modifications to the deformation field, which represents a significant advancement in deformable image registration. This feature proved invaluable to physicians engaged in adaptive radiation therapy planning, enhancing the robustness and applicability of the algorithm.

The optimization of the proportionality constant k served as an effective parameter for fine-tuning the performance of the GMM+Poisson algorithm. The value k=0.0075 was identified as the optimal proportionality constant that closely emulated the Fast Symmetric Forces DEMONS algorithm [30]. This optimized k enabled the deformation field to be modeled similarly to the DEMONS, as evidenced by the Dice Similarity Coefficient (DSC) metrics. Although FIG. 8 highlights instances where the GMM+Poisson algorithm failed to capture the tumor fully, even with these modifications, the overall performance surpassed that of the DEMONS method.

The comparative study involving DEMONS DIR provided significant insights into the applicability and limitations of the GMM+Poisson algorithm. The exemplary method exhibited superior performance in multiple cases, notably in avoiding including healthy tissue within contours, a critical concern in medical imaging [17], [32], [36]. DEMONS, although accurate, often suffer from false-positive inclusions of healthy tissue, mainly near bone structures. The differences in performance could be attributed to the underlying mathematical models; DEMONS employs an optical flow model or the forces can mix structures of a similar HU, whereas the exemplary method leverages Gaussian Mixture Models that segments tumor and healthy tissue regions.

The exemplary system and method provide potential breakthroughs in medical imaging for NSCLC. Through innovative algorithmic design, incorporation of cancer-specific attributes, and attention to practical requirements, this method marks a significant step toward enhancing the accuracy and utility of imaging techniques for NSCLC treatment.

The paradigm shift towards personalized medicine in radiation oncology necessitates the integration of cutting-edge technological advancements, such as ML and DIR. The treatment of NSCLC particularly benefits from these innovations, owing to the unique challenges posed by respiratory-induced anatomical variations [5], [10].

The exemplary method synergizes GMM and a modified Poisson equation to achieve efficient and accurate propagation of GTV contours across the diverse respiratory phases of a 4D-CT scan. Unlike previous methods [13], [14], [33], [35], the GMM+Poisson algorithm's combination of GMM clustering with a variable diffusion coefficient for the Poisson equation offers a nuanced representation of the lung's complex structures, resulting in more refined and robust tumor delineation.

The study's benchmarking against physician-generated contours propagated by DEMONS DIR method further emphasizes the robustness and efficiency of the exemplary method [37]. By successfully overcoming the movement-induced geometric uncertainties inherent in NSCLC cases and outpacing conventional DIR techniques, the GMM+Poisson algorithm provides a technical improvement to patient-centric treatment planning.

Overall, the GMM+Poisson algorithm offered an efficient, accurate and robust approach to propagate GTV contours across different respiratory phases in a 4D-CT scan. By incorporating machine learning and a modified Poisson equation, a more precise delineation of tumor boundaries was achieved, which can aid in clinical decision-making and treatment planning. The study results suggest that the proposed algorithm has the potential to be a useful tool and unveils unsupervised machine learning as a viable method for radiation therapy planning.

Finally, the exemplary system and method represents a significant stride in the radiotherapy workflow, showcasing the potential of unsupervised machine learning in radiation therapy planning. By facilitating more accurate tumor delineation and reducing treatment planning time, it aligns itself with the broader objective of delivering fast, reliable, robust, and patient-tailored solutions. It is contemplated that the exemplary system and method can be extend beyond NSCLC, fostering further exploration and refinement in other areas of oncology where precision and efficiency are paramount.

Machine Learning. The term “artificial intelligence” (e.g., as used in the context of AI systems) can include any technique that enables one or more computing devices or computing systems (i.e., a machine) to mimic human intelligence. Artificial intelligence (AI) includes but is not limited to knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders. The term “deep learning” is defined herein to be a subset of machine learning that enables a machine to automatically discover representations needed for feature detection, prediction, classification, etc., using layers of processing. Deep learning techniques include but are not limited to artificial neural networks or multilayer perceptron (MLP).

Machine learning models include supervised, semi-supervised, and unsupervised learning models. In a supervised learning model, the model learns a function that maps an input (also known as feature or features) to an output (also known as target or target) during training with a labeled data set (or dataset). In an unsupervised learning model, the model has a pattern in the data. In a semi-supervised model, the model learns a function that maps an input (also known as a feature or features) to an output (also known as a target) during training with both labeled and unlabeled data.

Neural Networks. An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers, such as an input layer, an output layer, and optionally one or more hidden layers. An ANN having hidden layers can be referred to as a deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tanH, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include but are not limited to backpropagation. It should be understood that an artificial neural network is provided only as an example machine learning model. This disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

A convolutional neural network (CNN) is a type of deep neural network that has been applied, for example, to image analysis applications. Unlike traditional neural networks, each layer in a CNN has a plurality of nodes arranged in three dimensions (width, height, and depth). CNNs can include different types of layers, e.g., convolutional, pooling, and fully connected (also referred to herein as “dense”) layers. A convolutional layer includes a set of filters and performs the bulk of the computations. A pooling layer is optionally inserted between convolutional layers to reduce the computational power and/or control overfitting (e.g., by down-sampling). A fully connected layer includes neurons, where each neuron is connected to all of the neurons in the previous layer. The layers are stacked similarly to traditional neural networks. GCNNs are CNNs that have been adapted to work on structured datasets such as graphs.

Other Supervised Learning Models. A logistic regression (LR) classifier is a supervised classification model that uses the logistic function to predict the probability of a target, which can be used for classification. LR classifiers are trained with a data set (also referred to herein as a “dataset”) to maximize or minimize an objective function, for example, a measure of the LR classifier's performance (e.g., an error such as L1 or L2 loss), during training. This disclosure contemplates that any algorithm that finds the minimum of a cost function can be used. LR classifiers are known in the art and are therefore not described in further detail herein.

An Naïve Bayes' (NB) classifier is a supervised classification model that is based on Bayes' Theorem, which assumes independence among features (i.e., the presence of one feature in a class is unrelated to the presence of any other features). NB classifiers are trained with a data set by computing the conditional probability distribution of each feature given a label and applying Bayes' Theorem to compute the conditional probability distribution of a label given an observation. NB classifiers are known in the art and are therefore not described in further detail herein.

A k-NN classifier is a supervised classification model that classifies new data points based on similarity measures (e.g., distance functions). The k-NN classifiers are trained with a data set (also referred to herein as a “dataset”) to maximize or minimize a measure of the k-NN classifier's performance during training. The k-NN classifiers are known in the art and are therefore not described in further detail herein.

A majority voting ensemble is a meta-classifier that combines a plurality of machine learning classifiers for classification via majority voting. In other words, the majority voting ensemble's final prediction (e.g., class label) is the one predicted most frequently by the member classification models. The majority voting ensembles are known in the art and are therefore not described in further detail herein.

Example Computing Device. It should be appreciated that the logical operations described above can be implemented in some embodiments (1) as a sequence of computer-implemented acts or program modules running on a computing system and/or (2) as interconnected machine logic circuits or circuit modules within the computing system. The implementation may be a matter of choice dependent on the performance and other requirements of the computing system. Accordingly, the logical operations described herein are referred to variously as state operations, acts, or modules. These operations, acts, and/or modules can be implemented in software, in firmware, in special purpose digital logic, in hardware, and any combination thereof. It should also be appreciated that more or fewer operations can be performed than shown in the figures and described herein. These operations can also be performed in a different order than those described herein.

In addition to the various systems discussed herein, FIG. 4 shows an illustrative computer architecture for a computing system 400 capable of executing the software components that can use the output of the exemplary method described herein. The computer architecture shown in FIG. 4 illustrates an example computer system configuration, and the computing system 400 can be utilized to execute any aspects of the components and/or modules presented herein described as executing on the analysis system or any components in communication therewith.

In an embodiment, the computing system 400 may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computing system 400 to provide the functionality of a number of servers that is not directly bound to the number of computers in the computing system 400. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or may be hired on an as-needed basis from a third-party provider. Some cloud computing environments may comprise cloud computing resources owned and operated by the enterprise as well as cloud computing resources hired and/or leased from a third-party provider.

In its most basic configuration, computing system 400 typically includes at least one processing unit 402 and system memory 410. Depending on the exact configuration and type of computing device, system memory 410 may be volatile (such as random-access memory (RAM)), non-volatile (such as read-only memory (ROM), flash memory, etc.), or some combination of the two.

The processing unit 404 may be a standard programmable processor that performs arithmetic and logic operations necessary for the operation of the computing system 400. While only one processing unit 404 is shown, multiple processors may be present. As used herein, processing unit and processor refers to a physical hardware device that executes encoded instructions for performing functions on inputs and creating outputs, including, for example, but not limited to, microprocessors (MCUs), microcontrollers, graphical processing units (GPUs), and application-specific circuits (ASICs). Thus, while instructions may be discussed as executed by a processor, the instructions may be executed simultaneously, serially, or otherwise executed by one or multiple processors. The computing system 400 may also include a bus or other communication mechanism for communicating information among various components of the computing system 400.

The computing system 400 is also shown to include a communications interface 420 that facilitates communications between computing system 400 and any external components or devices, including a user device 430. For example, communications interface 420 can provide means for transmitting data to, or receiving data from a user device. Accordingly, communications interface 430 can be or can include a wired or wireless communications interface (e.g., jacks, antennas, transmitters, receivers, transceivers, wire terminals, etc.) for conducting data communications, or a combination of wired and wireless communication interfaces. In some embodiments, communications via communications interface 420 are direct (e.g., local wired or wireless communications) or via a network (e.g., a WAN, the Internet, a cellular network, etc.). For example, communications interface 420 may include one or more Ethernet ports for communicably coupling computing system 400 to a network (e.g., the Internet). In another example, communications interface 420 can include a WiFi transceiver for communicating via a wireless communications network. In yet another example, communications interface 420 may include cellular or mobile phone communications transceivers.

In some implementations, computing system 400 is communicably coupled to user device 430 via communications interface 420 (e.g., via a wireless network). User device 430 may be a computing device including a memory (e.g., RAM, ROM, Flash memory, hard disk storage, etc.), a processor (e.g., a general-purpose processor, an application specific integrated circuit (ASIC), one or more field programmable gate arrays (FPGAs), a group of processing components, or other suitable electronic processing components), and a user interface (e.g., a touch screen), allowing a user to interact with computing system 400. User device 430 can include, for example, mobile phones, electronic tablets, laptops, desktop computers, workstations, vehicle dashboards, and other types of electronic devices. More generally, user device 430 may include any electronic device that allows a user to interact with computing system 400, and more broadly with computing system 400, by presenting and/or receiving user inputs through a user interface. In some implementations, user device 430 represents a user interface positioned on computing system 400 itself. In some implementations, user device 430 is configured to execute (i.e., run) a software application that presents the various user interfaces described herein.

In light of the above, it should be appreciated that many types of physical transformations take place in the computing system 400 in order to store and execute the software components presented herein. It also should be appreciated that the computing system 400 may include other types of computing devices, including hand-held computers, embedded computer systems, personal digital assistants, and other types of computing devices known to those skilled in the art. It is also contemplated that the computer architecture may not include all of the components shown in FIG. 4, may include other components that are not explicitly shown, or may utilize an architecture different than that shown.

In an example implementation, the processing unit 404 may execute program code stored in the system memory 410. For example, the bus may carry data to the system memory 410, from which the processing unit 404 receives and executes instructions.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language, and it may be combined with hardware implementations.

Moreover, the various components may be in communication via wireless and/or hardwire or other desirable and available communication means, systems and hardware. Moreover, various components and modules may be substituted with other modules or components that provide similar functions.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the nth 10 reference in the list. All references cited and discussed in this specification are incorporated herein by reference in their entireties and to the same extent as if each reference was individually incorporated by reference.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

### REFERENCES

[1] B. K. Horn and B. G. Schunck, “Determining optical flow,” Artificial intelligence, vol. 17, no. 1-3, pp. 185-203, 1981.

[2] J.-P. Thirion, “Image matching as a diffusion process: an analogy with maxwell's demons,” Medical image analysis, vol. 2, no. 3, pp. 243-260,1998.

[3] T. Kanai, N. Kadoya, K. Ito, Y. Onozato, S. Y. Cho, K. Kishi, S. Dobashi, R. Umezawa, H. Matsushita, K. Takeda et al., “Evaluation of accuracy of b-spline transformation-based deformable image registration with different parameter settings for thoracic images,” Journal of radiation research, vol. 55, no. 6, pp. 1163-1170, 2014.

[4] Y. Lei, Y. Fu, J. Harms, T. Wang, W. J. Curran, T. Liu, K. Higgins, and X. Yang, 4D-CT Deformable Image Registration Using an Unsupervised Deep Convolutional Neural Network. Artificial Intelligence in Radiation Therapy: Springer International Publishing, 2019, pp. 26-33. doi:10.1007/978-3-030-32486-5_4

[5] C. L. Brouwer, A. M. Dinkla, L. Vandewinckele, W. Crijns, M. Claessens, D. Verellen, and W. van Elmpt, “Machine learning applications in radiation oncology: Current use and needs to support clinical implementation,” Physics and Imaging in Radiation Oncology, vol. 16, pp. 144-148, 2020.

[6] S. Hindocha, K. Zucker, R. Jena, K. Banfill, K. Mackay, G. Price, D. Pudney, J. Wang, and A. Taylor, “Artificial intelligence for radiotherapy auto-contouring: Current use, perceptions of and barriers to implementation,” Clinical Oncology, vol. 35, no. 4, pp. 219-226, Apr. 2023. doi:10.1016/j.clon.2023.01.014

[7] L. A. Künzel and D. Thorwarth, “Towards real-time radiotherapy planning: The role of autonomous treatment strategies,” Physics and Imaging in Radiation Oncology, vol. 24, pp. 136-137, Oct. 2022. doi:10.1016/j.phro.2022.11.006

[8] H. Piperdi, D. Portal, S. S. Neibart, N. J. Yue, S. K. Jabbour, and M. Reyhan, “Adaptive radiation therapy in the treatment of lung cancer: An overview of the current state of the field,” Frontiers in Oncology, vol. 11, p. 5024, 2021.

[9] H. Onishi, H. Shirato, Y. Nagata, M. Hiraoka, M. Fujino, K. Gomi, K. Karasawa, K. Hayakawa, Y. Niibe, Y. Takai et al., “Stereotactic body radiotherapy (sbrt) for operable stage i non-small-cell lung cancer: can sbrt be comparable to surgery?” International Journal of Radiation Oncology* Biology* Physics, vol. 81, no. 5, pp. 1352-1358, 2011.

[10] M. Field, N. Hardcastle, M. Jameson, N. Aherne, and L. Holloway, “Machine learning applications in radiation oncology,” Physics and Imaging in Radiation Oncology, vol. 19, pp. 13-24, 2021. doi:10.1016/j.phro.2021.05.007

[11] U. Ricardi, S. Badellino, and A. R. Filippi, “What do radiation oncologists require for future advancements in lung sbrt?” Physica Medica, vol. 44, pp. 150-156, 2017.

[12] K. E. Rosenzweig, J. L. Fox, E. Yorke, H. Amols, A. Jackson, V. Rusch, M. G. Kris, C. C. Ling, and S. A. Leibel, “Results of a phase i dose-escalation study using three-dimensional conformal radiotherapy in the treatment of inoperable non-small cell lung carcinoma,” Cancer, vol. 103, no. 10, pp. 2118-2127, 2005. doi:10.1002/cncr.21007

[13] C. Zheng, X. Wang, J. Chen, Y. Yin, and D. Feng, “Deformable registration model with local rigidity preservation for radiation therapy of lung tumor,” in International Conference on Image Processing. IEEE, 2012, Conference Proceedings. doi:10.1109/icip.2012.6467199

[14] M. Peroni, M. F. Spadea, M. Riboldi, G. Baroni, G. T. Chen, and G. C. Sharp, “Validation of an automatic contour propagation method for lung cancer 4d adaptive radiation therapy,” in 2009 IEEE International Symposium on Biomedical Imaging: From Nano to Macro. IEEE, 2009, Conference Proceedings, pp. 1071-1074. doi:10.1109/isbi.2009.5193241

[15] M. Thompson and K. E. Rosenzweig, “The evolving toxicity profile of sbrt for lung cancer,” Translational lung cancer research, vol. 8, no. 1, p. 48, 2019.

[16] N. T. Sebastian, M. Xu-Welliver, and T. M. Williams, “Stereotactic body radiation therapy (sbrt) for early-stage non-small cell lung cancer (nsclc): contemporary insights and advances,” Journal of thoracic disease, vol. 10, no. Suppl 21, p. S2451, 2018.

[17] N. Samavati, M. Velec, and K. K. Brock, “Effect of deformable registration uncertainty on lung sbrt dose accumulation,” Medical physics, vol. 43, no. 1, pp. 233-240, 2016.

[18] H. Zhong, J. Kim, J. Gordon, S. Brown, B. Movsas, and I. Chetty, “Morphological analysis of tumor regression and its impact on deformable image registration for adaptive radiotherapy of lung cancer patients,” in World Congress on Medical Physics and Biomedical Engineering, June 7-12, 2015, Toronto, Canada. Springer, 2015, pp. 579-582.

[19] H. Zhong and I. J. Chetty, “Adaptive radiotherapy for nsclc patients: utilizing the principle of energy conservation to evaluate dose mapping operations,” Physics in Medicine & Biology, vol. 62, no. 11, p. 4333, 2017.

[20] C. L. Guy, E. Weiss, G. E. Christensen, N. Jan, and G. D. Hugo, “Caliper: A deformable image registration algorithm for large geometric changes during radiotherapy for locally advanced non-small cell lung cancer,” Medical physics, vol. 45, no. 6, pp. 2498-2508, 2018.

[21] J.-P. Thirion, “Fast non-rigid matching of 3d medical images,” Ph.D. dissertation, INRIA, 1995.

[22] H. Wang, L. Dong, J. O'Daniel, R. Mohan, A. S. Garden, K. K. Ang, D. A. Kuban, M. Bonnen, J. Y. Chang, and R. Cheung, “Validation of an accelerated ‘demons’ algorithm for deformable image registration in radiation therapy,” Physics in Medicine & Biology, vol. 50, no. 12, p. 2887, 2005.

[23] P. Cachier, X. Pennec, and N. Ayache, “Fast non rigid matching by gradient descent: Study and improvements of the “demons” algorithm,” Ph.D. dissertation, Inria, 1999.

[24] B. C. Lowekamp, D. T. Chen, L. Ibáñez, and D. Blezek, “The design of simpleitk,” Frontiers in neuroinformatics, vol. 7, p. 45, 2013.

[25] Z. Yaniv, B. C. Lowekamp, H. J. Johnson, and R. Beare, “Simpleitk image-analysis notebooks: a collaborative environment for education and reproducible research,” Journal of digital imaging, vol. 31, no. 3, pp. 290-303, 2018.

[26] U. Schneider, E. Pedroni, and A. Lomax, “The calibration of ct hounsfield units for radiotherapy treatment planning,” Physics in Medicine & Biology, vol. 41, no. 1, p. 111, 1996.

[27] J. Ehrhardt, R. Werner, A. Schmidt-Richberg, and H. Handels, “Statistical modeling of 4d respiratory lung motion using diffeomorphic image registration,” IEEE transactions on medical imaging, vol. 30, no. 2, pp. 251-265, 2010.

[28] R. Beare, B. Lowekamp, and Z. Yaniv, “Image segmentation, registration and characterization in r with simpleitk,” Journal of statistical software, vol. 86, 2018.

[29] T. Vercauteren, X. Pennec, A. Perchant, and N. Ayache, “Diffeomorphic demons using itk's finite difference solver hierarchy,” The Insight Journal, 11 2008.

[30] —, “Non-parametric diffeomorphic image registration with the demons algorithm,” in International Conference on Medical Image Computing and Computer-Assisted Intervention. Springer, 2007, pp. 319-326.

[31] X. Gu, H. Pan, Y. Liang, R. Castillo, D. Yang, D. Choi, E. Castillo, A. Majumdar, T. Guerrero, and S. B. Jiang, “Implementation and evaluation of various demons deformable image registration algorithms on a gpu,” Physics in Medicine & Biology, vol. 55, no. 1, p. 207, 2009.

[32] I. E. van Dam, J. R. v. S. de Koste, G. G. Hanna, R. Muirhead, B. J. Slotman, and S. Senan, “Improving target delineation on 4-dimensional ct scans in stage i nsclc using a deformable registration tool,” Radiotherapy and Oncology, vol. 96, no. 1, pp. 67-72, 2010.

[33] M. Guckenberger, K. Baier, A. Richter, J. Wilbert, and M. Flentje, “Evalution of surface-based deformable image registration for adaptive radiotherapy of non-small cell lung cancer (nsclc),” Radiation Oncology, vol. 4, pp. 1-13, 2009.

[34] D. Fabri, V. Zambrano, A. Bhatia, H. Furtado, H. Bergmann, M. Stock, C. Bloch, C. Lütgendorf-Caucig, S. Pawiro, D. Georg et al., “A quantitative comparison of the performance of three deformable registration algorithms in radiotherapy,” Zeitschrift für Medizinische Physik, vol. 23, no. 4, pp. 279-290, 2013.

[35] Mohanalin, P. K. Kalra, and N. Kumar, “Poisson's equation-based image registration: An application for matching 2d mammograms,” Journal of Medical Imaging and Health Informatics, vol. 4, no. 1, pp. 49-57, Mar. 2014. doi:10.1166/jmihi.2014.1220

[36] S. Oh and S. Kim, “Deformable image registration in radiation therapy,” Radiation Oncology Journal, vol. 35, no. 2, pp. 101-111, 2017. doi:10.3857/roj.2017.00325

[37] R. W. Underberg, F. J. Lagerwaard, B. J. Slotman, J. P. Cuijpers, and S. Senan, “Use of maximum intensity projections (mip) for target volume generation in 4dct scans for lung cancer,” International Journal of Radiation Oncology* Biology* Physics, vol. 63, no. 1, pp. 253-260, 2005.

What is claimed is:

1. A method for deformable image registration, the method comprising:
receiving at least one set of medical images for a patient, wherein at least one medical image comprises pre-drawn contours;
delineating (e.g., clustering) one or more regions of interest in the at least one set of medical images using a trained clustering machine learning algorithm;
determining deformed contours for the one or more regions of interest (e.g., tumor) in the at least one set of medical images using a finite element analysis algorithm over one or more of a plurality of phases of a physiological cycle (e.g., phases of breath cycle); and
outputting the deformed contours relative to the one or more regions of interest.

2. The method of claim 1, wherein delineating (e.g. clustering) one or more regions of interest in the at least one set of medical images using a trained clustering machine learning algorithm comprises:
initializing cluster parameters;
training a cluster machine learning algorithm using the at least one medical image comprising pre-drawn contours;
delineating a plurality of contours of the one or more regions of interest (e.g., clustering); and
clustering voxels of a medical scan image of the second set within the one or more regions of interest.

3. The method of claim 1, wherein the trained clustering machine learning algorithm comprises an unsupervised or supervised machine learning algorithm.

4. The method of claim 3, wherein the trained clustering machine learning algorithm comprises a Gaussian mixture model.

5. The method of claim 1, wherein determining deformed contours for the at least one set of medical images comprises:
deriving a deformation field using the finite element analysis algorithm, wherein the deformation field is dependent on an adjustable parameter (i.e., k); and
applying the deformation field over the delineated one or more regions of interest over one or more of a plurality of phases of the physiological cycle, thereby forming deformed contours.

6. The method of claim 5, the method further comprising receiving an adjusted value of the adjustable parameter from a user.

7. The method of claim 6, wherein in response to the user adjusting the adjustable parameter, the deformed contours are re-evaluated with the adjusted parameter (e.g. k′) and the re-evaluated deformed contours are output.

8. The method of claim 1, wherein the finite element analysis algorithm comprises a modified Poisson's equation, a modified Navier-Stokes equation, a modified Lagrangian mechanics-based algorithm, or an elastodynamic equation.

9. The method of claim 1, further comprising determining a dose distribution for one or more of the plurality of phases, wherein determining the dose distribution comprises:
determining a dose distribution for fewer than the plurality of phases;
deriving dose parameters from the fewer than the plurality of phases dose distribution; and
applying the dose parameters to deformed distribution for the plurality of phases.

10. The method of claim 1, further comprising determining a dose distribution for one or more of a plurality of phases comprises.

11. The method of claim 1, wherein medical images comprise 4D CT, 4D PET CT, MRI, ultrasound, radionuclide imaging, optical imaging.

12. The method of claim 1, wherein the deformation contours are subsequently used for diagnostic and/or treatment planning.

13. A system for deformable image registration, wherein the system comprises:
a user device comprising a means for input and output; and
one or more computing systems comprising one or more processors and one or more storage devices, wherein the one or more storage devices have instructions stored thereon, that when executed by the one or more processors, causes the one or more processors to perform a method, the method comprising:
receiving at least one set of medical images for a patient, wherein at least one medical image comprises pre-drawn contours;
delineating (e.g., clustering) one or more regions of interest in the at least one set of medical images using a trained clustering machine learning algorithm;
determining deformed contours for the one or more regions of interest (e.g., tumor) in the at least one set of medical images using a finite element analysis algorithm over one or more of a plurality of phases of a physiological cycle (e.g. phases of breath cycle); and
outputting the deformed contours relative to the one or more regions of interest.

14. The system of claim 13, wherein medical images comprise 4D CT, 4D PET CT, MRI, ultrasound, radionuclide imaging, optical imaging.

15. The system of claim 13, wherein delineating (e.g. clustering) one or more regions of interest in the at least one set of medical images using a trained clustering machine learning algorithm comprises:
Initializing cluster parameters;
training a cluster machine learning algorithm using the at least one medical image comprising pre-drawn contours;
delineating a plurality of contours of one or more regions of interest (e.g., clustering); and
clustering voxels of a medical image of the at least one set of medical images within the one or more regions of interest.

16. The system of claim 13, wherein the trained clustering machine learning algorithm is a Gaussian mixture model.

17. The system of claim 13, wherein determining deformed contours for the at least one set of medical images comprises:
deriving a deformation field using the finite element analysis algorithm, wherein the deformation field is dependent on an adjustable parameter (i.e., k); and
applying the deformation field over the delineated one or more regions of interest over one or more of a plurality of phases of the physiological cycle thereby forming deformed contours.

18. The system of claim 17, the method further comprising receiving an adjusted value of the adjustable parameter from a user.

19. The system of claim 18, wherein in response to the user adjusting the adjustable parameter, the deformed contours are re-evaluated with the adjusted parameter (e.g. k′) and the re-evaluated deformed contours are output.

20. The system of claim 13, wherein the finite element analysis algorithm comprises a modified Poisson's equation, a modified Navier-Stokes equation, a modified Lagrangian mechanics-based algorithm, or an elastodynamic equation.
